(19) United States
(12) Patent Application Publication
(10) Pub. No.: US 2014/0105959 A1
(43) Pub. Date: Apr. 17, 2014

(54) METHOD AND DEVICE FOR REDUCING DERMAL FILLER ADVERSE EVENTS

(71) Applicant: ALLERGAN, INC., IRVINE, CA (US)

(72) Inventors: Sumit Paliwal, Goleta, CA (US); Dennis Van Epps, Goleta, CA (US)

(73) Assignee: ALLERGAN, INC., IRVINE, CA (US)

(21) Appl. No.: 14/050,579

(22) Filed: Oct. 10, 2013

Related U.S. Application Data
(60) Provisional application No. 61/713,125, filed on Oct. 12, 2012.

(51) Int. Cl. A61K 31/137; A61K 9/70

(52) U.S. Cl. CPC A61K 31/137; A61K 9/7023
USPC 424/449; 514/653; 514/561; 514/682

(57) ABSTRACT

Methods and devices of aesthetically enhancing and improving appearance of aging skin are provided. Methods may include the steps of treating a skin site selected for dermal filler administration with a mechanism effective to enhance drug delivery across the skin, applying to the treated site, a device, compound or formulation including an active agent effective to reduce visible bruising due to administration of a dermal filler into the site, and administering a dermal filler into the skin site thereby reduce the appearance of wrinkles or folds in the skin site without causing significant bruising. Devices for reducing or ameliorating adverse events from dermal filler administration are also included.

This application claims the benefit of, and priority to U.S. Provisional Patent Application No. 61/713,125, filed Oct. 12, 2012, the entire disclosure of which is incorporated herein by this reference.

The present invention generally relates to dermal filler injectables and more specifically relates to a methods and devices for reducing dermal filler adverse events.

Over the last decade, use of hyaluronic acid (HA) as a dermal filler has gained rapid acceptance for facial rejuvenation applications such as treatment of wrinkles and correction of deeper facial folds. The tremendous growth in the use of HA filler products is due in large part to their lower cost, increased effectiveness, treatment longevity and favorable safety profile compared to their predecessor collagen-based filler products. However, regardless of the remarkable biocompatibility of HA with human skin, the highly aggressive and traumatic injection procedure sometimes leads to minor complications and adverse events after filler administration. Among the most common injection site adverse events include swelling, erythema, bruising, pain, and tenderness. Prolonged occurrence of these adverse events, particularly bruising, is highly undesirable for patients seeking faster times to social engagement after the treatment. Clinical studies suggest that as high as 80% of subjects receiving HA filler for nasolabial fold augmentation report bruising events. Bruises chiefly result from the puncturing and bleeding of skin vasculature during the intensive injection procedure. Since HA fillers used for dermal augmentation are typically under-saturated, excessive bleeding may cause the blood to be entrapped within the filler, leading to dark bruise spots that can last for extended periods. Clinical studies report that filler bruises often last for up to one week, causing significant social embarrassment and loss of work-time for the patients.

There is a substantial need in the industry for easy to use, effective methods and devices for reducing dermal filler adverse events, such as bruising. The present invention meets this need.

### SUMMARY

Accordingly, methods and device for reducing, for example, preventing or reducing a potential for, visible bruising following dermal filler administration in a patient are provided.

In one aspect of the invention, a method is provided generally comprising the step of applying to the skin site, for example, noninvasively or minimally invasively, for example, using diffusion through the skin, a device, compound or formulation, including an active agent effective to reduce or prevent significant visible bruising due to administration of an injectable dermal filler to the skin site.

In some embodiments, the device comprises a substrate, for example in the form of a dermal patch, and the active agent disposed on or in the substrate. For example, the patch may be substantially saturated with the active agent. In some embodiments, the patch is designed to cover a dermal filler treatment site, for along the line of injection of a dermal filler. The patch may be a patch about the size and shape of the skin site being treated. The device may be in the form of a patch containing a vasoconstricting agent or other active agent effective to reduce bruising in skin. The device may include the active agent in the form of a formulation such that the active agent is releasable from the patch in a sustained release manner when the patch is applied to the skin site. The active agent may be one or more of any number of active agents known as vasoconstricting agents.

Alternatively, or additionally the active agent may be selected from hemostatic drugs, coagulating agents, anti-inflammatory drugs, anti-histamine drugs, healing agents, analgesics, antioxidants, antiseptics and antibiotics. In this respect, although much of the present specification describes methods for treatment or prevention of visibly significant bruising resulting from dermal filler injection, in other aspects of the invention, the adverse event to be prevented or reduced, at least in intensity, may be an adverse event such as pain, inflammation, redness, itching, swelling and the like, and the active agent being an agent suitable for ameliorating such adverse event.

In a specific embodiment, the active agent is a vasoconstricting agent, for example, phenylephrine formulated in a range of about 100 ppm up to about 1000 ppm. Even more specifically, the device or compound may comprise phenylephrine at about 100 ppm, about 200 ppm, about 300 ppm, about 400 ppm, about 500 ppm or at about 600 ppm.

In another aspect of the invention, the active agent is a hemostatic agent, the hemostatic agent being selected from the group consisting of tranexamic acid and aminocaproic acid, or another agent known to have hemostatic properties when administered or applied to skin or tissue.

In some embodiments, the active agent is a blood coagulating agent, a vitamin K or is an agent selected from fibrinogen, microfibrillar collagen hemostat, and chitosan.

Turning now to another aspect of the invention, in some embodiments, the method may comprise the additional step of treating the skin site selected for dermal filler administration with a mechanism effective to enhance drug delivery across the skin, prior to the step of applying to the skin site the device, compound or formulation including an active agent.

For example, the mechanism to enhance drug delivery may comprise a chemical agent. In one embodiment, the chemical agent is an agent selected from the group of agents consisting of organic solvents, and/or surfactants (anionic, cationic, non-ionic and zwitterionic types), azones, fatty acids, and/or transcutol.

In another embodiment, the mechanism to enhance drug delivery is an electro-, or a mechanical-, and/or a thermal-device effective to treat the skin so as to enhance drug delivery across the skin surface and into the dermal layers, for example, at a depth of where visible bruising from dermal filler injection is likely to occur.

In a specific embodiment, the mechanism to enhance drug delivery comprises a mechanism effective to apply current to the skin site thereby utilizing electro-osmosis to transport both charged and neutral active agents into the skin.

In yet another embodiment, the mechanism effective to enhance drug delivery is a mechanical device comprising at least one of micron-sized needles, sonophoresis apparatus, laser-induced photo-mechanical waves, jet injectors and/or mechanical peeling of superficial skin layers with an adhesive or abrasive.

Methods of aesthetically enhancing and improving the appearance of aging skin are also provided. For example a skin treatment method is provided generally comprising the steps of treating a skin site selected for dermal filler administration with a mechanism effective to enhance drug delivery across the skin, and applying, minimally invasively or noninvasively, to the treated site, a device, compound or formulation, including an active agent effective to reduce visible bruising due to administration of a dermal filler into the site, as described elsewhere herein, and administering a dermal filler into the skin site thereby reduce the appearance of wrinkles or folds in the skin site without causing significant bruising. The transmission of the active agent across the outermost layers of the skin may be accomplished by means of diffusion, permeation, or absorption of the active agent, for example, through the stratum corneum into the deeper epidermis layers, in which the agent will reach cells and tissue at an effective therapeutic concentration.

In yet another aspect of the invention, a method for reducing bruising along a line of injection following dermal filler administration in a patient is provided, the method comprising treating a skin site selected for dermal filler administration with a mechanism effective to enhance drug delivery across the skin, and applying to the treated skin site, a device, compound or formulation including an active agent effective to reduce or prevent significant visible bruising along a line of injection due to administration of an injectable dermal filler to the skin site.

Each and every feature described herein, and each and every combination of two or more of such features, is included within the scope of the present invention provided that the features included in such a combination are not mutually inconsistent.

### BRIEF DESCRIPTION OF THE DRAWINGS

These and other aspects and advantages of the present invention may be more clearly understood by considering the following Detailed Description, in conjunction with the accompanying Drawings of which:

FIG. 1 shows photographs of rat skin in a test for effectiveness of the present invention to reduce the occurrence of bruising.

### DETAILED DESCRIPTION

Methods and device of reducing, for example, preventing or at least lessening the potential of, visible bruising following dermal filler administration in a patient are provided. The present methods and devices are especially advantageous to reduce the occurrence of bruising along the injection site which sometimes occurs for a temporary period, due to trauma to tissues following dermal filler administration, for example, for filling-in or correcting wrinkles, folds or creases in the skin.

In one aspect of the invention, a method is provided generally comprising the step of applying to the skin site, a device, compound or formulation, including an active agent effective to reduce or prevent significant visible bruising due to administration of an injectable dermal filler to the skin site. The device, compound or formulation may be applied to the skin site in a minimally invasive, or substantially non-invasive manner, for example, by causing diffusion of an active agent into the epidermis, rather than by injection with a conventional needle and syringe.

A device in accordance with one aspect of the invention for reducing dermal filler administration adverse events, for example, bruising along a line or region of subdermal injection, generally comprises a substrate configured to be applied to skin, for example, at a proposed injection site, and an active agent disposed on or in the substrate, the active agent being capable of reducing or even preventing significant visible bruising, for example, by constricting vessels in the skin and/or causing blanching upon contact with skin. The substrate may be configured to cover not only the point of injection, but also or alternatively, to cover the line of injection along a wrinkle, fold or crease being treated, for example.

In some embodiments, the devices and methods of the invention reduce visible bruising at least 50% or greater, relative to dermal filler administration without use of the device or method, or reduces the time period required for visible bruising to become insignificant or not visibly apparent, relative to dermal filler administration without use of the device. In some embodiments, the devices and methods reduce bruising, for example visible bruising, entirely, for example, 100%.

The present methods and devices may limit the intensity and/or occurrence of, and/or reduce recovery time of, at least one adverse event from dermal filler administration such as pain, inflammation, redness, itching, swelling, discoloration, etc.

In accordance with one embodiment, a device is provided in the form of a topical drug delivery patch, and a vasoactive pharmaceutical drugs can be included as a part of the topical drug delivery patch, or may be encompassed in a topical formulation containing at least one active pharmaceutical ingredient. Examples of suitable active pharmaceutical ingredients include vasoconstrictive drugs, hemostatic drugs, coagulating agents, anti-inflammatory drugs, anti-histamine drugs, healing agents, analgesics, antioxidants, antiseptics and antibiotics.

In a specific embodiment, the active agent is one or more of epinephrine, phenylephrine, pseudophenylephrine, or similar vasoconstrictor agent. In a specific embodiment, the agent is phenylephrine.

For example, devices of the invention may encompass a topical formulation containing at least one vasoactive pharmaceutical drug to limit the intensity and/or occurrence of bruising resulting from dermal filler administration.

Bruising from dermal filler administration is a type of hematoma that develops due to leakage of blood from blood vessels into the surrounding tissue due to trauma. The said vasoactive drug can be a pharmaceutical molecule that when introduced in the living tissue (skin and subcutaneous tissues such as fascia, fat, and muscle) is capable of transiently decreasing or stopping the leakage of blood from the vessels at the tissue site. Controlling the traumatic leakage of blood can be achieved by several mechanisms including constriction of blood vessels by vasoconstrictive drugs, and manipulating clotting of blood by hemostatic drugs. The said vasoactive agents in the device include, but not limited to, vasoconstrictive drugs (e.g., phenylephrine, epinephrine, adrenaline-analogs, drugs capable of stimulating α₁-adrenergic receptor, drugs acting on vascular smooth muscle cells leading to narrowing of blood vessels), and hemostatic drugs (such as antifibrinolytics drugs—tranexamic acid, aminocaproic acid, etc.; blood coagulation factors; vitamin K; fibrinogen; microfibrillar collagen hemostat; and chitosan).

In some embodiments, the active agent is a agent or drug selected from the group of agents consisting of .alpha.-agonists such as naphazoline, and tetrahydrozoline, sympathomimetics such as phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, albuterol, amphetamine, methamphetamine, benzphetamine, ephedrine, phenylpropanolamine, methentermine, phentermine, fenfluramine, propylhexedrine, diethylpropion, phenmetrazine, and phendimetrazine, adrenaline-analogs, an agent capable of stimulating α₁-adrenergic receptor, and an agent effective to act on vascular smooth muscle cells leading to narrowing of blood vessels.

In one embodiment, the formulation is a cream, gel, or substance which is applied to skin. The formulation can be applied by rubbing it on to the skin surface, preferably using a suitable applicator device. In other embodiments, the formulation is included in a topical patch to allow sustained release of said vasoactive drug from the patch in a controlled manner, as described elsewhere herein.

It may be desirable, in some embodiments in accordance with the invention, to apply said devices to the skin for a temporary period prior to dermal filler administration. The time period may be a time period of at least about one minute, up to about 24 hours. In an exemplary embodiment, the time period may be a time period between about 10 minutes, about 20 minutes about 30 minutes, up to one hour, prior to the dermal filler treatment. For optimal effect, the time period may vary depending on the specific vasoactive drug being applied, and is, for example, determined by the time taken for the vasoactive drug to be released from the device, diffuse inside the skin tissue to reach a desired potent concentration, and additionally, for example, the time taken by the vasoactive drug to interact with the tissue and produce intended beneficial effect.

In other instances, said devices may be applied to the skin for a temporary period following dermal filler administration. The time period may be a time period of at least about one minute, up to about 24 hours. In an exemplary embodiment, the time period may be a time period between about 10 minutes, about 20 minutes about 30 minutes, up to one hour, prior to the dermal filler treatment. As mentioned above, the time period may vary depending on the specific vasoactive drug being applied, and is, for example, determined by the time taken for the vasoactive drug to be released from the device, diffuse inside the skin tissue to reach a desired potent concentration, and additionally, for example, the time taken by the vasoactive drug to interact with the tissue and produce intended beneficial effect.

In yet other embodiments, the devices may be applied to skin during dermal filler administration, for example, along the line of intended injection (e.g. covering the entire wrinkle, fold, crease or other skin defect) but not covering the specific injection point. In this case, it may be beneficial if the device is configured to maintain visibility of the skin region being treated, for example, in order that the physician can view the wrinkle being treated as the needle or cannula is manipulated within the skin during the treatment. The device may therefor sometimes comprise a transparent or translucent patch.

In a specific experiment performed during development of the present invention, it took phenylephrine (formulated at 100 ppm in a topically placed cotton gauze) about 10 minutes to release, diffuse and cause significant vasoconstriction in hairless rat skin.

In certain embodiments, mechanisms are provided that are effective to actively enhance transport of vasoactive drugs across the skin (mechanisms will be referred to as “enhancers” in the rest of the invention). For example, in one embodiment of the invention, the method includes the step of treating a skin site selected for dermal filler administration with a mechanism effective to enhance drug delivery across the skin prior to treating the skin site with the device or compound containing the active agent effective to reduce or ameliorate the adverse event. For example, the mechanism, hereinafter sometimes referred to as “mechanical enhancer” may comprise a mechanical device capable of gently stripping of the superficial skin layers from the epidermis. For example, the mechanism may be an adhesive tape or abrasive surface. It has been discovered that use of such mechanisms increase phenylephrine diffusion through the skin and result in significant reduction in skin's vasoconstriction time (e.g. 5 minutes) by phenylephrine using the same patch device. In certain embodiments, it might be useful to apply the mechanism, enhancement device, after filler administration, preferably in devices where the vasoactive drug may interfere with safety and/or potency of fillers.

Enhancers either by themselves or in synergy with the device can facilitate delivery of vasoactive drug from the device into the skin. In an exemplary embodiment, chemicals capable of increasing skin's permeability such as organic solvents, surfactants (anionic, cationic, non-ionic and zwitterionic types), azones, fatty acids, and/or transcutol are included within the vasoactive drug formulation as an enhancer. The said chemical based enhancers interact with skin's superficial layer by either fluidizing or extracting skin lipids, and thereby, rendering skin permeable to vasoactive drug.

In alternative embodiments, enhancers are electro-mechanical-thermal devices included within the principal device containing the vasoactive drug formulation. The said electro-mechanical-thermal devices employ use of electrical, mechanical or thermal energy, or their combinations to increase skin permeability towards vasoactive drug. Example of devices using electrical energy include iontophoresis devices that employ small currents and electro-osmosis to transport both charged and neutral vasoactive drugs into the skin. Examples of mechanical devices include a patch of micron-sized needles, sonophoresis apparatus, laser-induced photo-mechanical waves, jet injectors and mechanical peeling of superficial skin layers with an adhesive or abrasive. Minimally-invasive ablation of the superficial skin with patches capable of producing intense but controlled thermal energy can also be included to safely enhance vasoactive drug transport. In some embodiments, the principal device carrying the vasoactive drug formulation is separate from a secondary device with enhancer, such that the skin is first pretreated with the said secondary device to increase skin permeability, followed by application of the said principal device for vasoactive drug delivery.

### Example 1

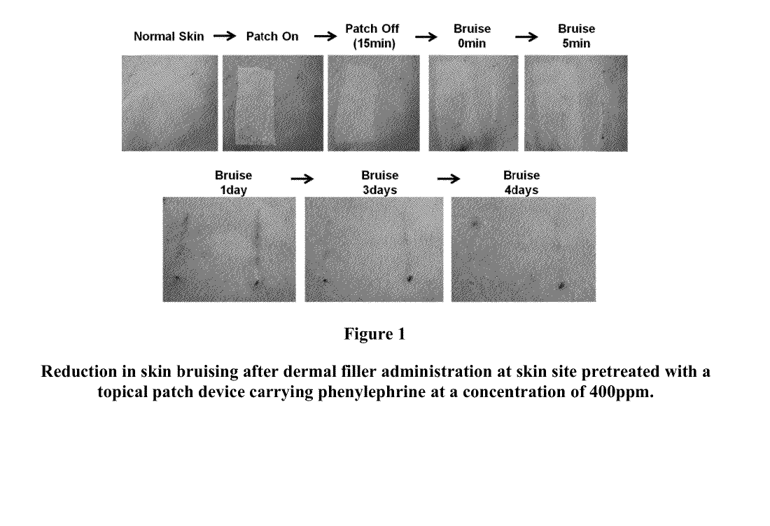

A device in accordance with embodiments of the invention, specifically, a patch consisting of a cotton fabric containing phenylephrine at a concentration of 400 ppm, was applied to the skin of a hairless rat (FIG. 1). Subsequent removal of the patch after 15 minutes showed significant vasoconstriction in skin regions lying directly underneath the patch. Administration of dermal filler at the vasoconstricted skin site and the adjacent untreated skin, yielded differential adverse events. Specifically, bruising at the vasoconstricted skin site, particularly along the line of injection, was lower than at the untreated skin site.

### Example 2

A 52-year old woman presents with significant signs of aging on her face, for example, her face exhibits deep nasolabial folds that she says make her look tired and stern. She is concerned that treatment with a dermal filler may result in temporary unsightly bruising, but her physician explains that there is a procedure which may reduce the possibility, and at least the severity, of this potential adverse event. She agrees to try the procedure. Her physician applies, along both nasolabial folds, a tool having a rolling head with numerous microneedles to penetrate the dermis with numerous punctures about one millimeter in depth. He then applies a phenylephrine-containing cotton-based patch, formulated to about 300 ppm phenylephrine, in accordance with the invention, on the skin along the left nasolabial fold, in a way that covers the entire injection site. The patch is left in place for about 7 minutes. The physician removes the patch and then carefully injects 0.6 cc of a HA-based dermal filler, specifically, Juvederm® XC, (available from Allergan, Inc., Irvine, Calif.) into the phenylephrine-treated dermis just beneath the fold line, using a 27 gauge needle. The treatment procedure is repeated for the right nasolabial fold, for example, using another identical patch and another 0.6 cc Juvederm® XC. Three days after the treatment procedure, the patient is pleased because she does not notice any visible bruising other than an insignificant, tiny red mark at each site where the dermal filler injection needle had entered the skin. One week after the procedure, the patient notices no bruising, nor red marks, and is pleased that the treatment makes her face appear younger, less tired and more friendly.

1. A method of reducing visible bruising following dermal filler administration in a patient, the method comprising:
treating a skin site selected for dermal filler administration with a mechanism effective to enhance drug delivery across the skin; and
applying to the treated skin site, a device, compound or formulation including an active agent effective to reduce or prevent significant visible bruising due to administration of an injectable dermal filler to the skin site.

2. The method of claim 1 wherein the step of applying is performed minimally invasively.

3. The method of claim 1 wherein the step of applying is performed by causing the active agent to be diffused through the skin.

4. The method of claim 1 wherein the device comprises a substrate and the active agent is disposed on or in the substrate.

5. The method of claim 4 wherein the substrate is a patch.

6. The method of claim 4 wherein the substrate is a patch and the patch is substantially saturated with the active agent.

7. The method of claim 4 wherein the substrate is a patch about the size and shape of the skin site being treated.

8. The method of claim 4 wherein the substrate is a patch about the size and shape of the skin site being treated and the active agent is a vasoconstricting agent.

9. The method of claim 1 wherein the device comprises a patch containing a vasoconstricting agent that is releasable from the patch in a sustained release manner when the patch is applied to the skin site.

10. The method of claim 9 wherein the vasoconstricting agent is phenylephrine formulated at a concentration range of about 100 ppm to about 1000 ppm.

11. The method of claim 10 wherein the phenylephrine is formulated at a concentration range of 300 ppm to 400 ppm.

12. The method of claim 1 wherein the mechanism to enhance drug delivery comprises a chemical agent.

13. The method of claim 12 wherein the chemical agent is an agent selected from the group of agents consisting of surfactants (anionic, cationic, non-ionic and zwitterionic types), azones, fatty acids, and/or transcutol.

14. The method of claim 1 wherein the mechanism effective to enhance drug delivery is an adhesive patch, which when placed on skin is capable of mechanically peeling superficial layers of skin.

15. The method of claim 1 wherein the mechanism effective to enhance drug delivery across skin is a micron-sized needle patch comprising needles of 20 μm to 1000 μm length, which when placed on skin is capable of puncturing skin, allowing drug to be delivered to the skin.

16. The method of claim 1 wherein the compound comprises phenylephrine at a concentration of about 100 ppm to about 400 ppm.

17. The method of claim 1 wherein the compound comprises phenylephrine at a concentration of about 400 ppm.

18. The method of claim 1 wherein the active agent is a hemostatic agent selected from the group consisting of tranexamic acid and aminocaproic acid.

19. The method of claim 1 wherein the active agent is a blood coagulating agent.

20. The method of claim 1 wherein the active agent is vitamin K.

21-24. (canceled)
